(19) United States
(12) Patent Application Publication
(10) Pub. No.: US 2003/0092671 A1
(43) Pub. Date: May 15, 2003

(54) Antithrombotic composition

(76) Inventors: Kristian Betton Johansen, Hellebaek (DK); Christian Frimundt Petersen, Koge (DK); Jack Hirsh, Burlington (CA); Jeffrey Weitz, Ancaster (CA)

Correspondence Address: BIRCH STEWART KOLASCH & BIRCH, PO BOX 747, FALLS CHURCH, VA, 22040-0747, US

(21) Appl. No.: 10/228,214

(22) Filed: Aug. 27, 2002

Related U.S. Application Data
(60) Provisional application No. 60/315,026, filed on Aug. 28, 2001.

(51) Int. Cl. A61K 31/737; A61K 31/727

(52) U.S. Cl. 514/54; 514/56

(57) ABSTRACT

The present invention relates to compositions and methods for the treatment of diseases or conditions characterised by excess thrombin generation or activity. In particular, the invention provides combination compositions comprising low molecular weight heparin (LMWH) and low molecular weight dermatan sulphate (LMWDS). Such compositions are well suited for the treatment of said diseases or conditions, and they exert an effect which is greater than sum of the effects of LMWH and LMWDS if dosed as single agents, i.e. the combination composition exerts a synergistic effect.

### FIELD OF INVENTION

The invention relates to methods and compositions for inhibiting or preventing thrombin generation or activity, and to the use of said compositions in the manufacture of medicaments.

### BACKGROUND OF THE INVENTION

Excessive generation of thrombin, which is a characteristic of many diseases including heart attack, stroke and deep vein trombosis, can be life threatening and requires treatment. One such treatment is administration of heparin, either unfractionated or low molecular weight heparin (LMWH) to patients. Heparin is a sulphated oligosaccharide belonging to the group of glycosaminoglycans (GAG). Heparin is not in itself an anticoagulant, but the effect is mediated through antithrombin (AT) and, to a lesser extent, heparin cofactor II (HCII). The use of heparin to treat the above mentioned diseases and conditions is limited by the risk of bleeding, especially in the lungs, CNS, and in the gastrointestinal system.

Another more recently suggested therapy for diseases characterised by excessive thrombin generation or activity is treatment with low molecular weight dermatan sulphate (LMWDS) as disclosed in e.g. WO 98/55514. Dermatan sulphate also belongs to the group of GAG and, like heparin, is not in itself an anticoagulant. The anticoagulant effect of dermatan sulphate is effected through HCII only, and as thrombin (factor IIₐ) is the exclusive target of HCII, dermatan sulphate is considered to be a selective inhibitor of thrombin.

Due to the above-mentioned risk of adverse effects (e.g. bleeding) in connection with heparin treatment, combination treatments including depolymerised heparin and unfractionated dermatan sulphate have been investigated [Cosmi, Thrombosis and Haemostasis, 70, 443-447, 1993]. However, only additive effects were found.

### SUMMARY OF THE INVENTION

It has now surprisingly been found that the combination of LMWH and LMWDS provides unexpectedly greater than additive, i.e. synergistic inhibitory effects on both fluid-phase and fibrin-bound thrombin compared to each of the compounds alone. The object of the invention is therefore to combine LMWH and LMWDS to maximise the anticoagulant effect of the two compounds while lowering the risk of adverse effects, e.g. bleeding.

Low molecular weight heparin and low molecular weight dermatan sulphate both exhibit anticoagulant activity, but they are believed to exert their activity through different mechanisms.

LMWH binds to antithrombin (AT) thereby increasing the inhibitory effect of AT towards the serine protease factor Xₐ, which is present in the plasma and plays an important role in the generation of thrombin, also referred to as factor IIₐ. LMWH thus inhibit the generation of thrombin. Depending on the length of the heparin oligosaccharide molecule, the heparin/AT complex may bind to thrombin, thereby directly inhibiting thrombin. A heparin oligosaccharide molecule has to contain 18 or more monosaccharide units to be able to bind to thrombin. As a consequence of the low molecular weight of LMWH it mainly contains oligosaccharides comprising less than 18 monosaccharide units, and the anticoagulant effect of LMWH thus predominantly relies on anti-factor Xₐ activity. LMWH also binds to HCII, potentiating its inhibitory effect towards thrombin.

LMWDS can inhibit fibrin-bound thrombin by activating HCII. In its activated conformation, the amino-terminal of HCII binds to exosite I on thrombin. Because thrombin also binds to fibrin via exosite I, activated HCII competes with fibrin for thrombin binding sites, displacing thrombin from fibrin. Displaced (i.e. fluid-phase) thrombin can then be inhibited by heparin/HCII, dermatan sulphate/HCII and heparin/AT.

Combining therapies which rely on different mechanisms to achieve maximum efficacy may improve tolerance to the therapy, and reduce the risk of side effects caused by high-dose and long-term use of the drugs in monotherapy. The combination of the invention may allow a reduction of the doses of each component required to obtain a therapeutic effect, and therefore also reduce the risk of adverse, toxic effects from each component. A lower dose may provide an increased safety margin relative to the margin for each component when dosed as single agents.

Accordingly, the invention relates to a pharmaceutical composition comprising at least one LMWH and at least one LMWDS, optionally together with a pharmaceutically acceptable excipient or vehicle.

In another aspect, the invention relates to a pharmaceutical composition comprising at least one LMWH and at least one LMWDS in separate containers and intended for simultaneous or sequential administration, optionally together with a pharmaceutically acceptable excipient or vehicle.

In a specific aspect of the invention, a pharmaceutical composition is provided comprising a combination of LMWH and LMWDS effective to exert a synergistic effect.

In a further aspect, the invention relates to a pharmaceutical composition comprising a unit dosage of at least one LMWH and a unit dosage of at least one LMWDS optionally together with a pharmaceutically acceptable excipient or vehicle.

In a preferred embodiment, the pharmaceutical composition contains amounts of LMWH and LMWDS that are at least 2-10 fold lower than the doses of each component required to prevent or inhibit thrombin activity or generation.

The abovementioned compositions also include pharmaceutically acceptable salts of LMWH and LMWDS, such as sodium, potassium, ammonia, magnesium and calcium salts.

In a still further aspect, the invention relates to a method of preventing or inhibiting thrombin generation or activity comprising administering to a patient in need thereof an effective amount of LMWH and LMWDS, either simultaneously or sequentially.

In a specific aspect of the invention a method of preventing or inhibiting thrombin generation is provided comprising administering to a patient in need thereof a LMWH and a LMWDS in an amount sufficient to provide synergistic activity.

In a still further aspect, the invention relates to the use of the above-mentioned method of treatment to prevent or ameliorate disease severity, disease symptoms, or periodicity or recurrence of a disease associated with excess thrombin generation or activity.

In a still further aspect, the invention relates to the use of LMWH and LMWDS, optionally together with a pharmaceutically acceptable excipient or vehicle, for the preparation of a medicament for the prevention or inhibition of thrombin generation or activity. In a preferred embodiment, the amounts of LMWH and LMWDS are so adjusted as to exert a synergistic effect.

### DETAILED DESCRIPTION OF THE INVENTION

Low Molecular Weight Heparin

In the present context, the term “low molecular weight heparin” (abbreviated to LMWH herein) refers to a mixture of oligosaccharides derived from heparin characterised by having AT and HCII related anticoagulant activity in vitro. The heterogeneous molecular structure of heparin consists mainly of repeating disaccharide units of uronic acid and N-sulphated glucosamine. The structure contains numerous variations of sulphation, L-epimerisation and N-deacetylation followed by N-sulphation. LMWH predominantly comprises oligosaccharides with less than 18 monosaccharide units, which are consequently too short to bind AT to thrombin.

The LMWH of the invention may be defined by one or more of the following characteristics:

a) antithrombin and heparin cofactor II related anticoagulant activity in vitro;

b) enriched for oligosaccharides containing less than 18 monosaccharide units;

c) having at least 10%, 15%, 20%, 25%, 30%, 35% or 40% oligosaccharides with at least one or more antithrombin binding pentasaccharide units;

d) enriched for oligosaccharides having a molecular weight of from about 1500 to about 5400 Da;

e) a peak molecular weight of about 3400 Da to 5600 Da;

f) a polydispersity of 1.1 to 1.8; and

g) an anti-factor Xₐ to anti-factor IIₐ ratio from 1.8 to 4;

In accordance with a particular aspect, a LMWH used in the present invention may have one of the following combinations of characteristics: a, b, c and d; a, b, c, and e; a, b, e, and f; a, b, e, and g; a, b, c, e, f, and g; b, c, d, and e; b, e, f and g; b, e, d and f; b, e, f, d and g; b, c, e, f and g; or a through g.

The term “enriched for oligosaccharides” is intended to indicate a fraction comprising at least 25%, 30%, 35%, 40%, 45% or 50% oligosaccharides within a specified molecular weight range, or with less than 18 monosaccharides units, respectively.

The term “antithrombin binding pentasaccharide unit” is intended to indicate a key structural unit of heparin consisting of three D-glucoseamine and two uronic acid residues as depicted in the structure below, wherein the central D-glucoseamine residue contains a unique 3-O-sulphate moiety:

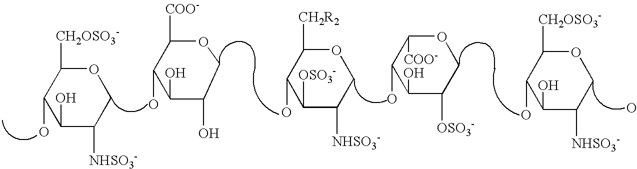

The pentasaccharide unit represents the minimum structure of heparin required for heparin to bind to AT [Choay, Biochem. Biophys. Res. Comm, 116, 492-499, 1983]. The binding of heparin to AT through the pentasaccharide unit results in conformational changes in the reactive centre loop in AT, which changes it from a weak to a strong inhibitor. As the LMWH of this invention is too short to bind the AT/heparin complex to thrombin, the main anti-coagulant effect of LMWH is mediated through factor Xₐ, resulting in inhibition of thrombin generation. Not all heparin molecules comprise the pentasaccharide structure, and those heparin molecules which do not, only exhibit anti-HCII activity.

By the term “unit dosage” is meant a unitary, i.e. a single dose which is capable of being administered to a patient, and which may be readily handled and packed, remaining as a physically and chemically stable unit dose comprising either the active material(s) as such or a mixture of it with solid or liquid pharmaceutical excipients.

It will be appreciated that it is possible to produce LMWH employed in this invention that has more particular characteristics than those set out in a) to g) above. For examples the LMWH may contain 5 to 17, e.g. 7 to 15, e.g. 9 to 13 monosaccharide units. It may even be possible to use an oligosaccharide which only contains the antithrombin binding pentasaccharide, i.e. 5 monosaccharide units. It may also be possible to employ LMWH enriched in oligosaccharides with a molecular weight from 1800 Da to 5100 Da; 2100 Da to 4800 Da; 2400 Da to 4500 Da; or 2700 to 4100 Da. It may also be possible to employ LMWH with a peak molecular weight from 3400 Da to 5600 Da; 3400 Da to 5000 Da; 3600 Da to 4800 Da or 3800 Da to 4600 Da. A LMWH employed in the present invention will not have similar anti-factor Xₐ and anti-factor IIₐ activity. In a preferred embodiment, the anti-factor Xₐ activity ranges from about 80 IU/mg to about 155 IU/mg, preferably from about 90 IU/mg to about 140 IU/mg. The anti-factor IIₐ activity ranges from about 10 IU/mg to 80 IU/mg, preferable from 20 IU/mg to 60 IU/mg.

A LMWH for use in the present invention may be obtained from animal tissues in a manner conventional for the preparation of such oligosaccharides of heparin. It may also be synthesised de novo, e.g. from the relevant monosaccharides.

By way of example, a LMWH may be obtained from unfractionated heparin by first depolymerising the unfractionated heparin to yield heparin with a lower molecular weight, and isolating or separating a LMWH fraction of interest. Commercial unfractionated heparin (e.g. USP or Ph. Eur.) is available from many sources, (e.g. SIGMA Chemical Co., St. Louis, Mo.), generally as an alkali metal or alkaline earth metal salt (most commonly as sodium heparin). Alternatively, the unfractionated heparin may be extracted from mammalian tissues or organs, particularly from intestinal mucosa or lungs from, for example cattle, swine and sheep, using a variety of methods known in the art and desribed in e.g. Coyne, Erwin, Chemistry and Biology of Heparin, Lundblads et al (Eds.), pp. 9-17, Elsevier, North-Holland, N.Y., 1981. In a preferred embodiment, the unfractionated heparin is extracted from porcine intestines.

Many processes for depolymerisation are known, and they are generally based on chemical or enzymatic breakdown of the heparin polymer. For instance, a LMWH employed in the present invention may be prepared from unfractionated heparin by benzylation followed by alkaline depolymerisation; nitrous acid depolymerisation; enzymatic depolymerisation; peroxidative depolymerisation, etc. For example, a LMWH employed in this invention may be prepared from unfractionated heparin using nitrous acid depolymerisation or periodate oxidation hydrolysis methods disclosed in WO 98/55515.

In a preferred embodiment, a LMWH employed in the present invention may be prepared from unfractionated heparin using heparinase mediated depolymerisation disclosed in U.S. Pat. No. 3,766,167 and U.S. Pat. No. 4,396,762. In particular, LMWH is prepared by controlled heparinase depolymerisation.

Commercially available LMWH include enoxaparin (e.g. Klexane® Aventis), tinzaparin (e.g. Innohep®, Leo Pharmaceutical Products), nadroparin (e.g. Fraxiparin®, Sanofi-Synthelabo), dalteparin (e.g. Fragmin®, Pharmacia) and ardeparin (e.g. Normiflo®, Wyeth Ayerst Laboratories).

Synthetic antithrombin binding pentasaccharide units may also be used in the methods and compositions of the present invention. For example, the commercially available pentasaccharide, including Fondaparinux (e.g. Arixtra®, Sanofi-Synthelabo and Organon) may be utilised.

Low Molecular Weight Dermatan Sulphate

In the present context, the term “low molecular weight dermatan sulphate” (abbreviated to LMWDS herein) refers to a mixture of oligosaccharides derived from dermatan sulphate characterised by having little or no AT related activity, but having HCII related anticoagulant activity in vitro. Dermatan sulphate consists of alternating uronic acid and N-acetylgalactosamine residues. Many glucuronic acid residues become epimerised at C-5 to yield iduronic acid residues. Subsequently, O-sulphation may occur at the C-4 or C-6 position of GaINAc or at the C-2 position of IdoA. LMWDS employed in this invention shows higher affinity towards HCII than native, unfractionated dermatan sulphate. LMWDS for use in the present invention may be defined by the following characteristics:

i) a sulphur content of 6.0% to 10.0% (w/w), e.g. from 6.0% to 8.0% (w/w), preferably from 6.5% to 8.0% (w/w);

ii) a sulphate/carboxyl ratio of 1.2 to 2.5, e.g. from 1.2 to 2.0, e.g. from 1.3 to 1.8, preferably from 1.3 to 1.6;

iii) a di-sulphated disaccharide content of 20% to 60% (w/w), preferably 30% to 60% (w/w) of the mono-sulphated disaccharide content; and

iv) an HCII mediated activity against thrombin in the range 20-60 IU/mg, preferably 30-60 IU/mg.

In a preferred embodiment of the invention, a LMWDS is selected that comprises a mixture of dermatan sulphate oligosaccharides with 90% or more having a molecular weight from about 1600 Da to about 20,000 Da, and a peak molecular weight from about 4500 Da to about 8000 Da. A preferred LMWDS is enriched for dermatan sulphate oligosaccharides with a molecular weight in the range of from about 5000 to about 8000 Da.

A LMWDS employed in this invention may be obtained from tissues in a manner conventional for the preparation of such oligosaccharides from unfractionated dermatan sulphate. It may also be synthesised de novo from the relevant monosaccharides. Preferably, a depolymerisation method that protects and facilitates the isolation of highly charged regions of dermatan sulphate is used to provide LMWDS for use in this invention with improved solubility and potency compared to unfractionated dermatan sulphate. By way of example, a LMWDS may be prepared by periodate oxidation, borohydride reduction, acid hydrolysis and ion exchange chromatography.

Sources of dermatan sulphate include mammalian tissues, for example, skin, including vascularised tissue and skin from porcine or bovine sources. Preferably, porcine intestinal mucosa are used as a source of dermatan sulphate.

Preferably, the present composition includes a LMWDS disclosed in WO 98/55514, which is incorporated herein by reference in its entirety. Methods of preparing such LMWDS also appear from WO 98/55514.

Compositions and Methods

The compositions and methods of the invention are useful in therapeutic applications for the prevention or treatment of conditions or diseases that are characterised by excess thrombin generation or activity and/or excess complement activation. Such conditions often occur where a subject has been exposed to trauma, for example in surgical patients. Trauma caused by wounds or surgery results in vascular damage and secondary activation of blood coagulation. These undesirable effects may occur after general or orthopaedic surgery, gynaecologic surgery, heart or vascular surgery, or other surgical procedures. Excess thrombin may also complicate progression of natural diseases, such as artherosclerosis which can cause heart attacks, stroke or gangrene of the limbs. Therefore, the methods and compositions of the present invention can be used to treat, prevent or inhibit a number of important cardiovascular complications, including unstable angina, acute myocardial infarction (heart attack), cerebral vascular accidents (stroke), pulmonary embolism, deep vein thrombosis, arterial thrombosis, etc.

In one aspect of the invention, methods and compositions are provided for preventing or inhibiting generation or activity of thrombin in patients at increased risk of developing a thrombus due to medical conditions that disrupt hemostasis (e.g. coronary artery disease, atherosclerosis, etc). In another aspect, methods and compositions are provided for patients at increased risk of developing a thrombus after a medical procedure, such as cardiac surgery, vascular surgery, or percutaneous coronary interventions. The compositions or individual oligosaccharide fractions in a method of the invention may be administered before, during or after the medical procedure.

Patients that may receive a treatment or be administered a composition of the present invention include animals, including mammalians, and particularly humans. Animals also include domestic animals, such as horses, cows, sheep, swine, cats, dogs, and zoo animals.

The composition of the present invention may be administered by any means that produce contact of the active agents with the active agent receptor site in the body of the patient. The LMWH and LMWDS can be administered simultaneously or sequentially in any order, and at different points in time, to provide the desired effect. It lies within the capability of a skilled physician or veterinarian to chose a dosing regime that optimises the effects of the compositions and treatments of the present invention. It is currently believed that the enhanced activity observed does not depend on the timing of the administration. The compositions may be administered in such oral dosage forms as tablets, capsules (each of which includes sustained release or times release formulations), pills, powders, granules, elixirs, tinctures, suspensions, syrups and emulsions. They may also be administered intravenous (bolus or infusion), intraperitoneal, subcutaneous, or in intramuscular form, all using dosage forms well known to those of ordinary skill in the art of pharmacy. The compositions of the invention may be administered in intranasal form via topical use of suitable intranasal vehicles, or via transdermal routes, for example using conventional transdermal skin patches. The dosage administration in a transdermal delivery system will be continuous rather than intermittent throughout the dosage regime.

The present invention includes combination treatments providing synergistic activity or delivering synergistically effective amounts of LMWH and LMWDS. Pharmaceutical compositions suited for use in the present invention include compositions wherein the active ingredients are present in a synergistically effective amount. By “synergistic activity” or “synergistically effective amount” is meant that a sufficient amount of LMWH and LMWDS will be present in order to achieve a desired result that is greater than the additive result achieved with each component on its own, e.g. improved inhibition of thrombin when treating a thrombus-related cardiovascular condition, such as those described above, for example, improved inactivation of clot-bound thrombin, improved inhibition of thrombin generation by catalysing factor Xₐ inactivation by antithrombin, etc.

The dosage regimen of the invention will vary depending upon known factors such as the pharmacodynamic characteristics of the particular agent and its mode and route of administration, the species, age, sex, health, medical condition, and weight of the patient, the nature and extent of the symptoms, the kind of concurrent treatment, the frequency of treatment, the renal and hepatic function of the patient, and the desired effect. The effective amount of a drug required to prevent, counter, or arrest progression of a disease or condition can be readily determined by an ordinarily skilled physician or veterinarian.

Typically, the active agents, i.e. LMWH and LMWDS, will each be present in the pharmaceutical composition at a concentration ranging from about 2 mg per dose to 1000 mg per dose and, more preferably, at a concentration ranging from 5 mg per dose to 500 mg per dose. Daily dosages can vary widely, but will usually be at concentrations ranging from about 20 mg per dose per day to about 100 mg per dose per day for each of the active components.

The composition of the present invention and components thereof typically comprise suitable pharmaceutical diluents, excipients or vehicles selected in accordance with the intended form of administration, and consistent with conventional pharmaceutical practices. The vehicle may be adapted to provide a synergistically effective amount of the active components to inhibit or prevent thrombin generation in a patient.

Suitable pharmaceutical vehicles are described in several standard textbooks, e.g. Remington, The Science and Practice of Pharmacy, 20^{th} Ed., Mack Publishing Company, 2000. By way of example, for oral administration in the form of a capsule or a tablet, the active components may be combined with a non-toxic, pharmaceutically acceptable inert vehicle such as lactose, starch, sucrose, methyl cellulose, magnesium stearate, glucose, calcium sulphate, dicalcium phosphate, mannitol, sorbitol, and the like. For oral administration in liquid form, the drug components may be combined with any oral, non-toxic, pharmaceutically acceptable inert vehicle such as ethanol, glycerol, water, and the like. Suitable binders (e.g. gelatin, starch, corn sweeteners, natural sugars including glucose, natural and synthetic gums, and waxes), lubricants, (e.g. sodium oleate, sodium stearate, magnesium stearate, sodium benzoate, sodium acetate and sodium chloride), disintegrating agents (e.g. starch, methyl cellulose, agar, bentonite and xanthan gum), flavouring agents, and colouring agents may also be combined in the compositions or components thereof.

Formulations for parenteral administration of the composition of the invention include aqueous solutions, syrups, aqueous or oil suspensions and emulsions with edible oil, such as cottonseed oil, coconut oil or peanut oil. Suitable dispersing or suspending agents for aqueous suspensions include synthetic or natural gums, such as tragacanth, alginate, acacia, dextran, sodium carboxymethylcellulose, gelatin, methycellulose and polyvinylpyrrolidone.

For parenteral administration, the composition of the invention may include a sterile aqueous or non-aqueous solvent, in particular water, isotonic saline, isotonic glucose solution, buffer solution or other solvent conveniently used for parenteral administration of therapeutically active compounds. The composition may be sterilised by, for instance, filtration through a bacteria retaining filter, addition of sterilising agent to the composition, irradiation of the composition, or heating the composition.

Alternatively, the compounds of the present invention may by provided as a sterile, solid preparation, e.g. a freeze-dried powder, which is readily dissolved in sterile solvent immediately prior to use.

The composition intended for parenteral administration may further comprise conventional additives such as stabilisers, buffers, or preservatives, e.g. antioxidants such as methylhydroxybenzoate or the like.

In addition to the formulations described previously, the compositions can also be formulated as a depot preparation. Such long-acting formulations may be administered by implantation (e.g. subcutaneously or intramuscularly) or by intramuscular injection. Thus, for example, the components of the present invention may be formulation with suitable polymeric or hydrophobic materials (for example as an emulsion in a pharmaceutically acceptable oil), or ion exchange resin, or as sparingly soluble derivatives, for example, as a sparingly soluble salt.

The composition of the invention and components thereof may comprise soluble polymers as targetable drug carriers.

The present invention also includes methods of using the compositions of the invention with one or more additional therapeutic agents including, without limitation, anti-platelet or platelet inhibitory agents such as aspirin, prioxicam, clopidogrel, ticlopidine, or glycoprotein IIb/IIIa receptor antagonists, thrombin inhibitors such as boropeptides, hirudin or argatroban; or thrombolytic or fibrinolytic agents, such as plasminogen activators (such as tissue plasminogen activator), anistreplase, urokinase, or streptokinase or combinations thereof.

The properties and characteristics of the LMWH and LMWDS used in the present invention may be determined using the following methods

1. Molecular weight by GPC-HPLC according to the method of Dedem, Pharmeuropa, 3, 202-218, 1991.

2. Sulphur content according to Ph. Eur. 2.ed, V.3.5.3.

3. Sulphate/carboxylate ratio according to Ph. Eur. 1997:0828.

4. HCII mediated antithrombin activity by a chromogenic assay (Diagnostica Stago, France) in a plasma free system with the 4. International Heparin Standard (code no. 82/502) as standard.

5. Anti-factor Xₐ and anti-factor IIa activity according to Ph. Eur. 1997:0828, both methods modified using the statistical methods for slope-ratio assays.

In addition to being useful in pharmaceutical compositions for the treatment of the cardiovascular conditions described above, one of skill in the art will readily appreciate that the active products can be used as reagents for elucidating the mechanisms of blood coagulation in vitro.

The invention will be described in the following, non-limiting example.

### EXAMPLE 1

Antithrombotic Activity of LMWH and LMWDS in Combination

An extracorporeal circuit was used to compare the anti-thrombotic activity of LMWH, LMWDS, and the combination of LMWH and LMWDS. As described in Weitz, Circulation, 99, 682-689, 1999, LMWH, LMWDS, or combinations thereof were added to recalcified human whole blood and maintained at 37° C. in a water bath. A peristaltic pump was then used to circulate the blood through a 40μ blood filter. Clotting of blood within the filter was detected by measuring pressure proximal to the filter with an in-line pressure gauge. The experiment ran for 90 minutes, and consequently the maximum achievable time to filter failure is >90 minutes. The LMWDS used in this investigation has the following characteristics: Mₚ 5000 Da, M_{w} 7600 Da, and polydispersity 1.4. The LMWH was the commercially available enoxaparin (Aventis), characterised by a peak molecular weight, Mₚ between 3500 Da and 5500 Da and anti-factor Xₐ activity of 90-125 IU/mg.

As illustrated in table 1, when used alone a LMWDS concentration of 200 μg/ml maintains filter patency for 65 minutes. Table 1 also illustrates that enoxaparin alone at 20 μg/ml maintains filter patency for 70 minutes. Finally, Table 1 illustrates that by combining LMWDS at 50 μg/ml and enoxaparin at 2 μg/ml, filter patency is maintained for 85 minutes. From these data, it is evident that a pronounced synergistic effect is obtained by combining LMWH and LMWDS. When LMWH and LMWDS are used in combination, the two drugs are effective at a less than 4 to 10 fold lower doses than those needed if the drugs were administered alone (i.e. 50 mg vs 200 mg/ml and 20 vs 2 mg/ml).

TABLE 1

GAG

| Enoxaparin | LMWDS | Time to filter failure |
|---|---|---|
| μg/ml | μg/ml | Minutes |
| 10 | — | 25 |
| 20 | — | 70 |
| — | 200 | 65 |
| 2 | 50 | 85 |

1. A pharmaceutical composition comprising a combination of
A) at least one heparin oligosaccharide fraction defined by one or more of the following characteristics
a) having antithrombin and heparin cofactor II related anticoagulant activity in vitro;
b) enriched for oligosaccharides containing less than 18 monosaccharide units;
c) having at least 15%, 20%, 25%, 30%, 35% or 40% oligosaccharides with at least one or more antithrombin binding pentasaccharide units;
d) enriched for oligosaccharides having a molecular weight of about from 1500 to about 5400 Da;
e) a peak molecular weight of from about 3400 Da to about 5600 Da;
f) a polydispersity of 1.1 to 1.8; and
g) an anti-factor Xₐ to anti-factor IIₐ ratio from 1.8 to 4; and
B) at least one dermatan sulphate fraction defined by the following characteristics
i) a sulphur content of 6.0 to 10.0% (w/w);
ii) a sulphate/carboxyl ratio of 1.2 to 2.5;
iii) a disulphated disaccharide content of 20% to 60% (w/w); and
iv) an HCII mediated activity against thrombin in the range 20-60 IU/mg, optionally together with a pharmaceutically acceptable excipient or vehicle.

2. A pharmaceutical composition comprising, in separate containers and intended for simultaneous or sequential use,
A) at least one heparin oligosaccharide fraction defined by one or more of the following characteristics
a) having antithrombin and heparin cofactor II related anticoagulant activity in vitro;
b) enriched for oligosaccharides containing less than 18 monosaccharide units;
c) having at least 15%, 20%, 25%, 30%, 35% or 40% oligosaccharides with at least one or more antithrombin binding pentasaccharide units;
d) enriched for oligosaccharides having a molecular weight of from about 1500 to about 5400 Da;
e) a peak molecular weight of from about 3400 Da to about 5600 Da;
f) a polydispersity of 1.1 to 1.8; and
g) a anti-factor Xₐ to anti-factor IIₐ ratio from 1.8 to 4; and
B) at least one dermatan sulphate fraction defined by the following characteristics
i) a sulphur content of 6.0 to 10.0% (w/w);
ii) a sulphate/carboxyl ratio of 1.2 to 2.5;
iii) a disulphated disaccharide content of 20% to 60% (w/w); and
iv) an HCII mediated activity against thrombin in the range 20-60 IU/mg, optionally together with a pharmaceutically acceptable excipient or vehicle.

3. A pharmaceutical combination composition comprising a unit dosage of
A) at least one heparin oligosaccharide fraction defined by one or more of the following characteristics
a) having antithrombin and heparin cofactor II related anticoagulant activity in vitro;
b) enriched for oligosaccharides containing less than 18 monosaccharide units;
c) having at least 15%, 20%, 25%, 30%, 35% or 40% oligosaccharides with at least one or more antithrombin binding pentasaccharide units;
d) enriched for oligosaccharides having a molecular weight of from about 1500 to about 5400 Da;
e) a peak molecular weight of from about 3400 Da to about 5600 Da;
f) a polydispersity of 1.1 to 1.8; and
g) a anti-factor Xₐ to anti-factor IIₐ ratio from 1.8 to 4; and
B) at least one dermatan sulphate fraction defined by the following characteristics
i) a sulphur content of 6.0 to 10.0% (w/w);
ii) a sulphate/carboxyl ratio of 1.2 to 2.5;
iii) a disulphated disaccharide content of 20% to 60% (w/w); and
iv) an HCII mediated activity against thrombin in the range 20-60 IU/mg, optionally together with a pharmaceutically acceptable excipient or vehicle.

4. A composition according to any of claims 1-3 wherein the heparin oligosaccharide fraction has the following characteristics a, b, c and d; a, b, c, and e; a, b, e, and f; a, b, e, and g; a, b, c, e, f, and g; b, c, d, and e; b, e, f and g; b, e, d and f; b, e, f, d and g; b, c, e, f and g or a through g.

5. A composition according to any of claims 1 to 4 wherein the dermatan sulphate oligosaccharide fraction has a molecular weight of from about 1600 Da to about 20,000 Da and a peak molecular weight of from about 4500 Da to about 8000 Da.

6. A composition according to any of claims 1-5 wherein the amounts of A) and B) are effective to exert a synergistic effect in preventing the generation or activity of thrombin.

7. A composition according to claim 6 wherein the doses of the heparin oligosaccharide fraction and dermatan sulphate oligosaccharide fraction are at least 2-10 fold lower than the doses of each fraction required to prevent or inhibit thrombin generation or activity if dosed alone.

8. A composition according to any of claims 1-7 wherein the heparin oligosaccharide fraction is the antithrombin binding pentasaccharide unit.

9. A method of preventing or inhibiting thrombin generation or activity in a patient comprising administering to a patient in need thereof an effective amount of a composition according to any of claims 1-8.

10. A method according to claim 9 wherein the heparin oligosaccharide fraction and dermatan oligosaccaride fraction are administered simultaneously or separately.

11. Use of a composition according to any of claims 1-8 for the preparation of a medicament for the prevention or inhibition of thrombin generation or activity.

12. The use according to claim 11 wherein the amounts of LMWH and LMWDS are effective to exert a synergistic effect.
